(19) United States
(12) Patent Application Publication
(10) Pub. No.: US 2004/0181194 A1
(43) Pub. Date: Sep. 16, 2004

(54) Instrument for cleaning and relieving obstructions in feeding tubes

(76) Inventor: Kathy S. Perkins, LaFollette, TN (US)

Correspondence Address: PITTS AND BRITTIAN P C, P O BOX 51295, KNOXVILLE, TN, 37950-1295, US

(21) Appl. No.: 10/388,789

(22) Filed: Mar. 14, 2003

(51) Int. Cl. A61M 25/00; B08B 9/04

(52) U.S. Cl. 604/267; 15/104.16; 15/104.2; 134/8

(57) ABSTRACT

A tube cleaning apparatus for insertion through a feeding tube inserted within a patient. The apparatus includes an elongated member having a manipulated end and an insertion end sized for insertion through the feeding tube during insertion in the patient. The insertion end includes a resilient material disposed radially thereon, including a plurality of filaments and/or a compressible material. The resilient material provides contact against interior surfaces of the feeding tube during insertion therethrough. A limit member is removably clamped around the elongated member at any position between the manipulated end and the insertion end, providing a maximum limit for insertion of the insertion end through the feeding tube. A method of cleaning is disclosed for utilizing an elongated member having a limit member clamped thereon for cleaning interior surfaces of a feeding tube positioned within a patient.

### CROSS REFERENCE TO RELATED APPLICATIONS

Not applicable.

### STATEMENT REGARDING FEDERALLY SPONSORED RESEARCH OR DEVELOPMENT

Not Applicable.

### BACKGROUND OF THE INVENTION

1. Field of Invention

This invention pertains to a cleaning device for tubes used for delivery of nourishment and/or medicine into patients, and more particularly, pertains to a device and method for cleaning obstructions within a feeding tube inserted into a patient.

2. Description of the Related Art

Feeding tubes are utilized by inserting a tube of sufficiently small diameter into a patient requiring assistance with the intake of liquid nourishment and/or medicine. A feeding tube for use over a relatively short time frame (i.e. days), is inserted through the patient's mouth or through the nasopharyngeal passageway, and can be extended through the esophagus and into the stomach for delivery of liquid nourishment and/or medicine into the patient. A feeding tube utilized over a long time frame (i.e. weeks), includes a percutaneous endogastric (PEG) tube that is inserted through an incision in the skin of the abdomen for extension through an incision in the stomach wall and positioning the feeding tube discharge end into the stomach and proximal to the small intestine. Alternatively, nutrient containing mixtures including dextrose, water or normal saline, and medications are administered by an intravenous (IV) feeding tube inserted through the skin and positioned into the lumen of a vein.

When a feeding tube is positioned into the patient's body, it is preferred that the tube not be removed for periodic cleaning of materials that become deposited on the interior wall surfaces of the feeding tube. If periodic cleaning is not accomplished during long-term use of the feeding tube while inserted into the patient, the removal and replacement of the feeding tube is recommended every two to four weeks to avoid build-up of obstructing materials that can occlude the tube.

As disclosed in U.S. Pat. No. 5,964,004, to Bean, a prior device for cleaning endoscopic tubes includes an elongated body having a socket at one end on which a multi-bladed cleaner unit is attached. The blades of the multi-bladed cleaner unit provide for scraping of the interior of an endoscopic tube. During cleaning, the scraping action of the blades may scour the tube interior and provide abraded interior surfaces on which additional obstructing materials can accumulate.

The prior devices are typically limited for cleaning by insertion through tubes that are removed from the patient's body to allow access during cleaning to both tube ends for pulling and pushing the tube cleaning device through the tube. In addition, prior tube cleaning devices have lacked an adjustable member for limiting the depth of insertion of a tube cleaning device inserted through a feeding tube positioned in the patient's body. There exists a need for an improved tube obstruction cleaner that is insertable through a feeding tube while the feeding tube is positioned in a patient for in vivo cleaning of the tube interior without requiring removal of the feeding tube.

### BRIEF SUMMARY OF THE INVENTION

A tube cleaning apparatus is disclosed for insertion through an interior of a fluid transfer tube inserted into a patient to provide for periodic cleaning of the tube interior. The tube cleaning apparatus includes an elongated member having an insertion end and a manipulated end. The elongated member is sized in diameter for insertion into the fluid transfer tube inserted into the patient. The insertion end includes a resilient material extending from a perimeter of the insertion end. The resilient material extends an adequate distance from the perimeter of the insertion end to provide for contact against the interior surface of the fluid transfer tube during insertion of the insertion end through the length of the fluid transfer tube. The resilient material is selected from a group of materials including, but not limited to, a resiliently compressible foam material, a plurality of filaments radially extended from the insertion end, and a self-supporting sponge material having filaments extended therefrom. A limit member is removably clamped at any of a plurality of positions along an axial length of the elongated member between the manipulated end and the insertion end. The limit member provides an adjustable limit for the insertion of the insertion end through the interior of the fluid transfer tube. A method is disclosed for cleaning an interior of a fluid transfer tube while retained within a patient by utilizing an elongated tube member having an insertion end configured to extend through the fluid transfer tube in cooperation with utilizing a limit member positioned on the elongated tube member to limit the depth of insertion of the tube member.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1a is a perspective view of a tube cleaning apparatus including an elongated tube member of the present invention;

FIG. 1b is a cross-section view along 1b-1b of FIG. 1a;

FIG. 2 is a side view of the elongated tube member inserted into a discharge end of a feeding tube;

FIG. 3a is an end view of the insertion limit member that is removably clamped on the elongated tube member;

FIG. 3b is a perspective view of the insertion limit member in an open configuration;

FIG. 4a is a side view of an embodiment of the insertion end of the elongated tube member of the present invention;

FIG. 4b is a side view of an alternative embodiment of the insertion end of the elongated tube member;

FIG. 4c is a side view of an alternative embodiment of the insertion end of the elongated tube member;

FIG. 5 is a side view of the tube cleaning apparatus inserted into a feeding tube disposed through the nasal passageway and positioned in the stomach of a patient; and

FIG. 6 is a side view of the tube cleaning apparatus inserted through a percutaneous endogastric tube inserted through a patent's abdomen and positioned in the stomach of the patient.

### DETAILED DESCRIPTION OF THE INVENTION

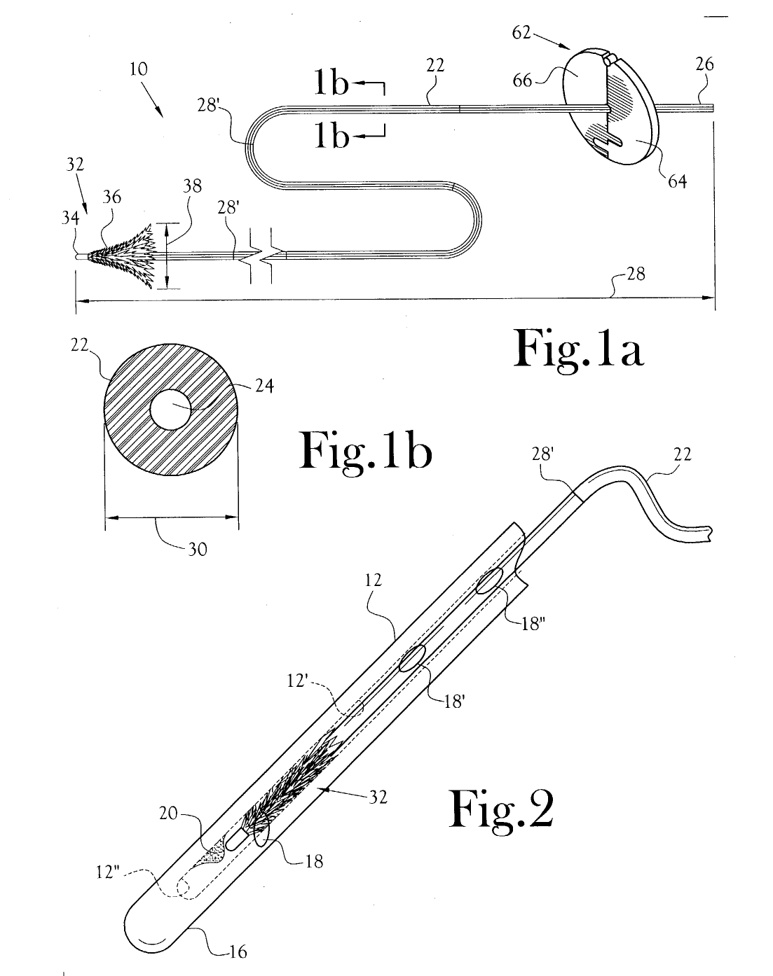

As illustrated in FIG. 1a, one embodiment of a tube cleaning apparatus 10 of the present invention is disclosed for insertion into a fluid transfer tube such as a feeding tube 12. The tube cleaning apparatus 10 includes an elongated tube member 22 having an insertion end 32 of a sufficient length from a first end 26 for insertion of the insertion end 32 through the interior length of the feeding tube 12 positioned into the stomach 90 of a patient 80. The insertion end 32, when positioned through the feeding tube 12, provides for in vivo cleaning of the interior 12′ of the feeding tube 12. The operator manipulates the first end 26 in order to position the insertion end 32 into the feeding tube distal end 16 (see FIG. 2). A means for cleaning extends from the insertion end 32 (see FIGS. 4a-4c) for a sufficient axial length to provide engagement of the means for cleaning against the walls of the feeding tube interiorly proximal to the distal interior surfaces 12″ of the feeding tube 12. An obstruction 20 and/or deposits of biological materials within the interior 12′ are contacted by the means for cleaning when the insertion end 32 is positioned in the feeding tube distal end 16, thereby dislodging the obstruction 20 and/or deposits for passage out of the interior of the feeding tube 12.

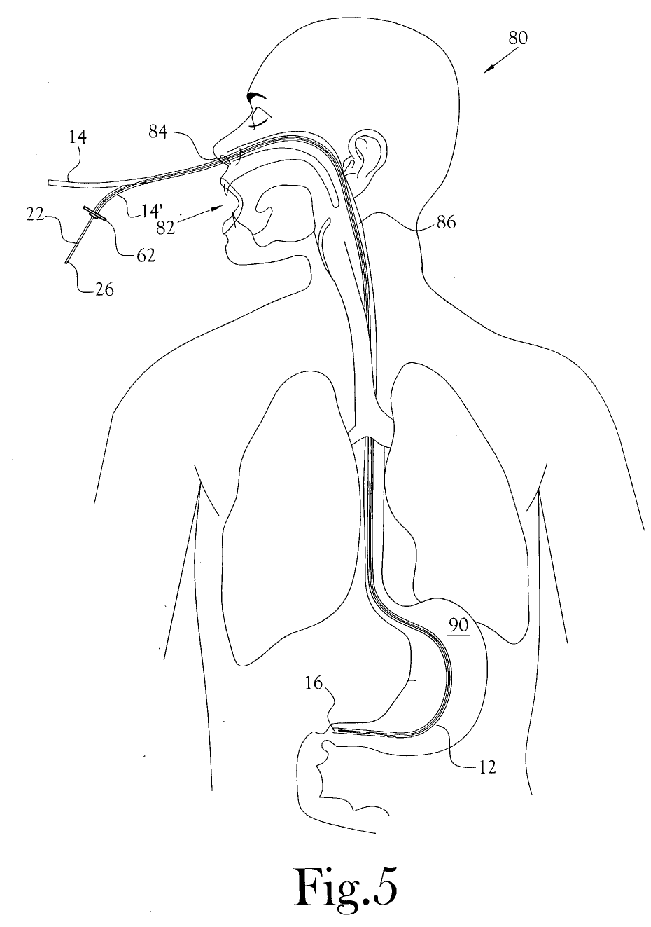

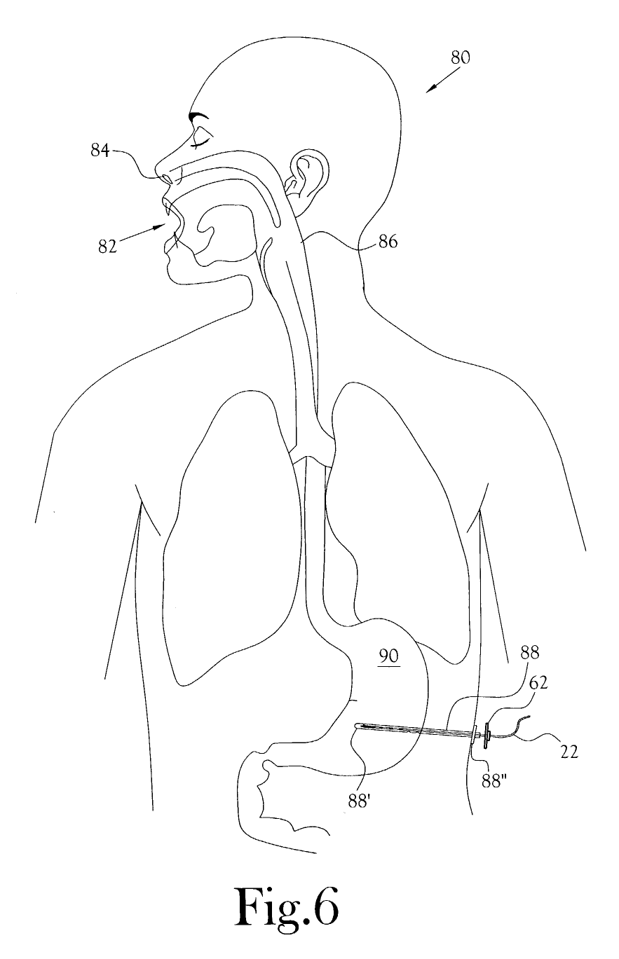

The tube member 22 includes a sufficient axial length, illustrated at 28 in FIG. 1a, extending between the insertion end 32 and the first end 26 to allow the first end 26 to remain outside of the feeding tube connection end 14 when the insertion end 32 is positioned within the feeding tube distal end 16. The first end 26 is gripped and manipulated by the operator when the insertion end 32 is extended into a feeding tube 12. Periodic cleaning of the feeding tube distal end 16 is required during positioning in the patient's stomach 90 when an obstruction 20 (see FIG. 2) occurs in the tube discharge portion of the feeding tube distal end 16. The tube discharge portion includes at least one discharge hole 18. A preferred embodiment for the tube discharge end includes a plurality of discharge holes 18, 18′, 18″ oriented in spaced apart configuration along the circumference and the length proximal of the tube distal end 16. To facilitate cleaning of a feeding tube 12 positioned in the patient's stomach (see FIGS. 5 and 6), the length 28 of the tube member 22 is selected from a range of lengths of about 40 inches (about 103 cm) to about 65 inches (about 167 cm) measured from the insertion end 32 to the manipulated first end 26. A preferred length 28 is about 50 inches (about 128 cm) to about 60 inches (about 154 cm). A length 28 of about 60 inches to about 65 inches is utilized for insertion of the tube member 22 through a nasopharyngeal feeding tube 12 (see FIG. 5). A length 28 of about 40 inches to about 50 inches is utilized for insertion of the tube member 22 through a PEG tube 88 (see FIG. 6), or a similar tube inserted through an incision in the abdominal wall. The tube member diameter illustrated at 30 in FIG. 1b is selected from a range of about 2.5 mm to about 4.5 mm, with a preferred diameter being about 3 mm. However, other diameters could be utilized. The tube member 22 is preferably composed of a flexible polymer material that is laterally bendable but is not significantly extendable in an axial length dimension of the tube member 22.

The length 28 preferably includes a plurality of increments 28′ etched into, or marked in permanent ink, on the diameter 30 in a plurality of equally spaced-apart locations (see FIG. 1a). The plurality of increments 28′ include increments of length of every ten cm, or, in the alternative, increments of every 12 inches, beginning with zero at the insertion end 32. The depth of insertion of the tube member 22 into the feeding tube 12 is quickly ascertained by observing the etched or marked increments 28′ of length on the diameter 30 of the tube member 22 that remains extended from the feeding tube connection end 14.

One embodiment of the tube member 22 includes a hollow core 24 extended through approximately the center of the axial length of the tube member 22 (see FIG. 1b). The hollow core 24 extends to about the distal end 34 (see FIG. 4a), and includes an internal diameter of about 1.0 mm. The hollow core 24 provides additional lateral flexibility for the tube member 22 that is preferably composed of a flexible polymer material. The additional lateral flexibility provided by the hollow core 24 allows the tube member 22 to be maneuvered through a feeding tube 12 that may have numerous bends and internal constrictions along its path through the esophagus 86 to the stomach 90 (see FIG. 5).

One embodiment of the insertion end 32 is illustrated in FIGS. 1a, 2, and 4a. The insertion end 32 includes a pliable elongated tip 34″ having a cylindrical configuration with a diameter of about 3.0 mm. The elongated tip 34″ is preferably composed of a polymer material and includes a length illustrated at 34′ in FIG. 4a of between about 0.5 cm to about 1.0 cm extended from the distal end 34. The elongated tip 34″ is flexible and is utilized to probe and dislodge an obstruction 20 by axially applied physical contact applied by the tip 34″ during manipulation of the first end 26 by an operator. The hollow core 24 extends to about the interior of the elongated tip 34″ of the tube member 22. Extended radially from the perimeter of the distal end 34 is a means for cleaning disposed proximal to the distal end 34. The means for cleaning includes a plurality of flexible brush filaments 36 positioned within an end segment having an axial length illustrated at 40 in FIG. 4a of about 3.0 cm. The plurality of flexible brush filaments 36 are shaped in a conical configuration, having base ends attached to the perimeter of the distal end 34 of the insertion end 32. The filaments 36 have distal ends extending radially from the insertion end 32, including short filaments of about 0.5 cm in length originating at a distance of about 1.0 cm spaced apart from the tip of the distal end 34. Filaments having a longer length of about 1.5 cm form a conical base portion having a diameter illustrated at 38 in FIG. 4a of between about 3.0 cm to about 3.5 cm. The conical configuration of the filaments 36 along the insertion end 32 provides for contact by a plurality of the short filaments and long filaments 36 against an obstruction 20 disposed on the walls bounding the interior 12′ of a feeding tube 12 having an interior diameter of between about 1.0 cm to about 3.5 cm. Alternative lengths for the plurality of filaments 36 can be readily provided for permanent attachment to the insertion end 32 for cleaning narrow internal diameter or wide internal diameter feeding tubes 12.

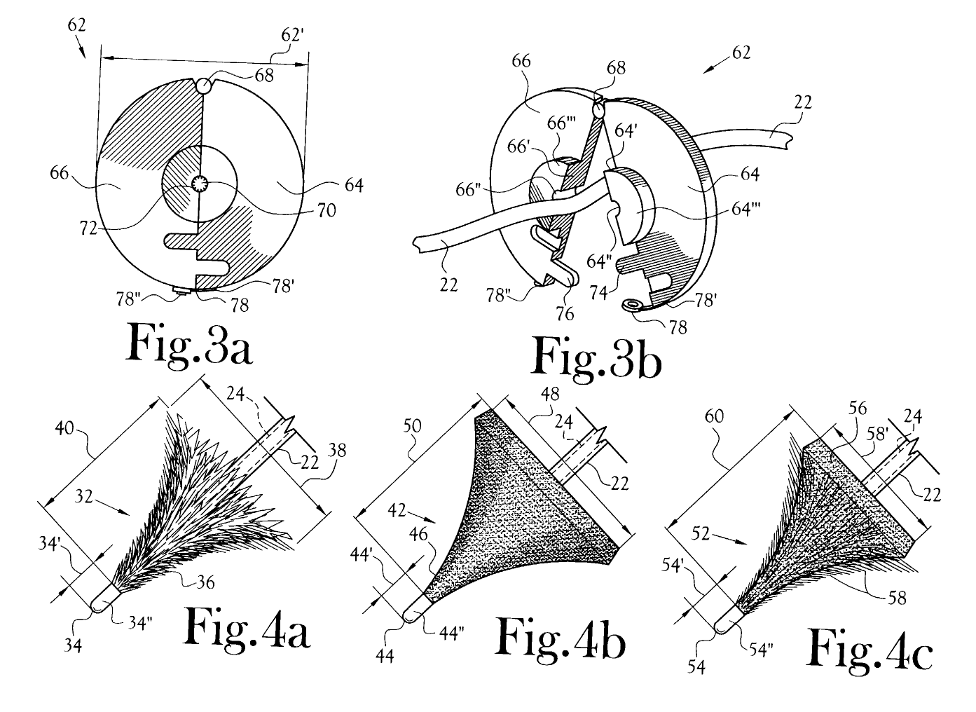

FIGS. 3a and 3b illustrate an insertion limit member 62, that is releasably clamped at any of a plurality of positions along the axial length 28 of the tube member 22. The limit member 62 includes a disc having two half segments 64, 66 that are connected by means for pivoting such as a hinge 68 positioned at one abutting end of each half segment 64, 66. The hinge 68 provides for the pivoting and closure of a leading edge 64′ of the first half segment 64 against a leading edge 66′ of the second half 66 to form a disc-shaped limit member 62 when closed. Each half segment 64, 66 includes a raised portion 64′″, 66′″ disposed on one face of each half segment 64, 66. The raised portions 64′″, 66′″ provide an additional depth of about one cm to the depth of about two cm of each half segment 64, 66. A central hole 70 in the closed limit member 62 is formed by one semi-circular opening 64″ in the leading edge 64′ of the first half segment 64 and the first raised portion 64′″, being aligned with an adjacently oriented semi-circular opening 66″ in the leading edge 66′ of the second half segment 66 and the second raised portion 66′″. The two aligned semi-circular openings 64″, 66″ are sized to accept a typical diameter of tube member 22 therein when the first half segment 64 and the second half segment 66 are closed and clamped around a portion of the tube member 22 (see FIGS. 1a and 3a). In order to assure a constricting fit of the two aligned semi-circular openings 64″, 66″ around the tube member 22, a plurality of flexible teeth 72 protrude inwardly of each interior circumference of each semi-circular opening 64″, 66″. Proper closure of the first and second half segments 64, 66 is assisted by alignment of a first arcuate segment 74 in the connecting edge 64′ of the first half segment 64, with a mirror-image shaped second arcuate segment 76 in the connecting edge 66′ of second half segment 66. When clamped in a position on a length of tube member 22 exterior of the feeding tube 12, the limit member 62 provides a limit to the depth of insertion of the insertion end 32 into an interior of the feeding tube 12.

The first and second half segments 64, 66 are maintained in a clamped configuration (see FIG. 3a) by a hinged means for connecting such as a clasp 78 hingedly attached to an exterior edge of the first half segment 64. The clasp 78 pivots about a hinged pivoting connection 78′ to allow clasp 78 to clamp onto a clasp receiving post 78″ positioned on an exterior edge of the second half segment 66 (see FIG. 3b). A preferred use of the limit member 62 includes clamping the limit member 62 onto the tube member 22 at about the length increment 28′ that coincides with the calculated length of insertion of the feeding tube 12 within the patient 80. The outer diameter 62′ of a closed limit member 62 is between about 2 cm to about 5 cm, and may have a preferred diameter 62′ of about 4 cm. The closed and clamped limit member 62 remains outside the connection end 14 of the feeding tube 12 due to the outer diameter 62′ being larger than the outer diameter of a typical feeding tube 12. The limit member 62, when properly positioned, prevents the tube member 22 from being inserted deeper than the calculated length of insertion of the discharge end 16 into the patient 80. The presence of the clamped limit member 62 prevents the tip of the distal end 34 from being forced through a discharge hole 18, or through the discharge end 16 of the feeding tube 12 when positioned in the patient's stomach 90.

One embodiment for positioning of the tube member 22 into a feeding tube 12 is illustrated in FIG. 5. A nasogastric feeding tube 12 is inserted through the patient's nasal passageway 84, through the esophagus 86 and into the stomach 90 of the patient 80. The insertion end 32 is inserted into the feeding tube 12 through the feeding tube connection end 14 that remains external of the patient 80. The connection end 14 typically includes at least one bifurcated valve 14′ for delivery of nutrients and/or medication in liquid form. During intermittent release of nutrients and/or medication fluids from the holes 18, 18′, 18″ in the tube discharge end 16, the distal interior surfaces 12″ of the tube discharge end 16 can accumulate biological material from the interaction of stomach fluids with the nutrients and/or medication fluids. Significant accumulation of one or more obstructions 20 such as deposits of biological material within the tube discharge end 16 can diminish the flow of nutrients and/or medication fluids into the patient's stomach 90. Therefore, periodic cleaning of the tube discharge end 16 by an attendant is typically required. It is a preferred method of cleaning for use of the tube member 22 for cleaning the feeding tube 12 while positioned in the stomach 90. Discharged biological materials and dislodged obstructions 20 are disposed into the stomach 90 for degradation by the stomach fluids and ingestion by the patient 80.

An alternative embodiment for positioning of a feeding tube in a patient 80 is illustrated in FIG. 6. A PEG tube 88 or a similar feeding tube is utilized for long-term feeding (i.e. for weeks and months), and is inserted through the patient's abdomen by means of a valve 88″ inserted through a dermal incision in the abdomen for extension through an internal incision in the stomach wall. The PEG tube discharge end 88′ is extended to be positioned into the stomach 90, or is further extended to be positioned through the pylorus and into the duodenum. A preferred insertion length for the tube member 22 is selected to equal a calculated length of insertion for the PEG tube 88 by estimating the actual depth of insertion of the PEG tube 88 utilizing information contained in the medical chart records provided for each patient. An alternative length and diameter for the tube member 22 may be selected for insertion through any other fluid transfer tube sized for insertion into the patient, including a tube selected from the group of a mouth feeding tube, a tube inserted through the throat, a tube inserted through the abdomen and into the stomach proximal of the small intestine, or an alternative tube known to those skilled in the art for conveying nutrients and/or medication fluids into a patient 80.

An alternative embodiment of the tube member 22 includes an insertion end 42 illustrated in FIG. 4b. The insertion end 42 includes a flexible elongated tip 44″ composed of a polymer material such as a medical grade plastic polymer. The tip 44″ extends from the distal end 44 for a length illustrated at 44′ of between about 0.5 mm to about 1.0 mm. The elongated tip 44″ is flexible and is utilized to probe and dislodge an obstruction 20 by axially applied physical contact applied by the tip 44″ during manipulation of the first end 26 by an operator. The hollow core 24 extends to about the interior of the elongated tip 44″ of the tube member 22. The hollow core 24 provides flexibility to allow the elongated tip 44″ and the insertion end 42 to be applied against the interior of the feeding tube 12 regardless of whether an obstruction 20 is positioned along a curve or along a generally straight portion of the feeding tube 12. Originating proximal of the elongated tip 44″ is a means for cleaning disposed to radially extend from the perimeter of the tube member 22 for a selected length illustrated at 50 in FIG. 4b of about 3.0 cm to about 3.5 cm spaced from the distal end 44. The means for cleaning includes a porous and resiliently compressible foam material 46. A sponge material may be utilized to form a conical shape having a narrow diameter of about 1 cm proximal to the distal end 44. The resilient material 46 increases in diameter to an outer diameter illustrated at 48 in FIG. 4b of about 3.0 cm to about 3.5 cm. Alternative outer diameters for the resilient material 46 can be selected for cleaning large diameter tubes.

An additional embodiment of the tube member 22 includes an insertion end 52 illustrated in FIG. 4c. The insertion end 52 includes a flexible elongated tip 54″ composed of a polymer material such as a medical grade plastic polymer. The elongated tip 54″ extends from the distal end 54 for a length illustrated at 54′ of between about 0.5 mm to about 1.0 mm. The elongated tip 54″ is flexible and is utilized to probe and dislodge an obstruction 20 by axially applied physical contact applied by the tip 54″ during manipulation of the first end 26 by an operator. The hollow core 24 extends to about the interior of the elongated tip 54″ of the tube member 22. The hollow core 24 provides flexibility to allow the elongated tip 54″ and the insertion end 52 to be applied against the interior of the feeding tube 12 regardless of whether an obstruction 20 is positioned along a curve or along a generally straight portion of the interior of the feeding tube 12. Originating proximal of the elongated tip 54″ is a means for cleaning disposed to extend from the perimeter surface of the tube member 22 a selected length illustrated at 60 in FIG. 4c of about 3.0 cm to about 3.5 cm from the distal end 54. The means for cleaning includes a plurality of resiliently bendable filaments 58 that are radially extended from a porous and resiliently compressible foam material 56, such as a sponge material. The combination of the foam material 56 and the filaments 58 forms a conical shape increasing from a narrow diameter of about 1.0 cm proximal of the distal end 54, to an outer diameter illustrated at 58′ in FIG. 4c of between about 3.0 cm to about 3.5 cm for cleaning biological materials from the tube interior 12′ of a broad range of diameters for feeding tubes 12.

A method of cleaning is disclosed for cleaning an interior 12′ of a feeding tube 12 having a discharge end 16 positioned within a patient. The method includes selecting an elongated tube member 22 having an appropriate tube member length 28 to allow insertion of the insertion end 32 through the length of the feeding tube 12 for positioning into the interior of the discharge end 16 of the feeding tube 12. The appropriate tube member length 28 is calculated by an operator to provide a sufficient length of the elongated tube member 22 for inserting, extending, and positioning of the insertion end 32 through the feeding tube 12 and into the interior of the discharge end 16. The method includes positioning the insertion end 32 through the valve port 14′ of the feeding tube 12 inserted into the patient 80 (see FIG. 5). An alternative step of positioning includes inserting the insertion end 32 into the abdominal valve 88″ of the PEG tube 88 inserted into the patient 80 (see FIG. 6). The method includes extending the insertion end 32 through the length of the feeding tube 12 but not further than the interior of the discharge end 16 of the feeding tube 12. The step of extending is accomplished by the operator manipulating the first end 26 of the elongated tube member 22 that remains external of the feeding tube 12. Upon extending the insertion end 32 proximal of the discharge end 16 and adjacent an obstruction 80, cleaning and dislodging of the obstruction 80 is accomplished by the operator's manipulation of the first end 26 in rotating movements and/or inserting and retracting movements. Any one of the disclosed configurations of the insertion ends 32, 42 or 52 is utilized for cleaning the interior 12′ of the feeding tube 12 with filaments 36 or 58, and/or resilient material 46 or 56. The method for cleaning is also utilized for cleaning an interior of a PEG tube 88 by inserting any one of the disclosed configurations of the insertion ends 32, 42 or 52 through the interior of the PEG tube 88.

The method of cleaning further includes selecting a limit member 62 that is removably clamped on the portion of the elongated tube member 22 that remains outside of the connection end 14 of the feeding tube 12. The outer and inner diameters of the disc-shaped limit member 62 are selected to provide the inner diameter of a central hole 70 to fit around the outer diameter 30 of the elongated tube member 22. An outer diameter 62′ of the limit member 62 is a sufficient diameter to contact against the exterior diameter of the connection end 14 of the feeding tube 12, thereby limiting the insertion of the elongated tube member 22 to no more than the length of insertion of the feeding tube 12 into the patient. With the limit member 62 clamped at an appropriate position on external diameter of the tube member 22, the steps of positioning and extending the insertion end 32 into the feeding tube 12 are restricted to a maximum length of insertion of the elongated member 22 in order to position the insertion end 32 within the discharge end 16 of the feeding tube 12 positioned within the patient's stomach 90.

The method of cleaning further includes removing the elongated tube member 22 from the interior of the feeding tube 12, or from the interior of the PEG tube 88. Upon removal of the elongated tube member 22, a selected volume of about 30 cc of fluid such as air is injected through the feeding tube 12. An alternative step of injecting includes flushing the feeding tube 12 with a selected volume of fluid of about 50 cc of water, saline, and/or saline containing nutrients. The selected volume of fluid, whether air or liquid, provides only a minimal additional volume of fluid injected into the patient's stomach 90.

From the foregoing description, it will be recognized by those skilled in the art that the elongated tube member 22 and the limit member 62 provide a tube cleaning apparatus guided by an operator through a feeding tube 12 inserted within a patient. The operator manipulates the first end 26 of the elongated tube member 22 during insertion of the distal insertion end 32 through the interior 12′ of the feeding tube 12 to provide positioning of the insertion end 32 proximal to an obstruction 20 within the feeding tube 12. A benefit provided by the tube cleaning apparatus 10 includes a flexible length 28 having a plurality of length increments 28′ etched or marked thereon, thereby providing a convenient visual check concerning the depth of penetration of the insertion end 32 into the feeding tube 12. It is preferable that an operator of the tube member 22 have a marker to indicate the depth of penetration so that the insertion end 32 is not extended through a nutrient discharge hole 18 in the distal end 16 of the feeding tube 12. An additional benefit of the tube cleaning apparatus 10 includes the limit member 62 that is clamped at any position along the length 28 of the elongated tube member 22. A preferred use of the limit member 62 includes clamping the limit member 62 on the exterior diameter of the elongated tube member 22 at any one of the plurality of positions along the tube member 22. The position selected by the operator is substantially equal to one of the length increments 28′ that coincides with the calculated insertion length of the feeding tube 12 into the patient's stomach 90. The clamped limit member 62 prevents the insertion end 32 from being inserted through the feeding tube 12 past a maximum depth of insertion that is substantially equal to the depth of insertion of the feeding tube 12 into the patient, thereby preventing the insertion end 32 from being forced against, or through the distal end 16 of the feeding tube 12 when positioned in the patient's stomach 90.

While the present invention has been illustrated by description of several embodiments and while the illustrative embodiments have been described in considerable detail, the scope of the invention is to be limited only by the appended claims and their legal equivalents. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of general inventive concept disclosed herein.

Having thus described the aforementioned invention, I claim:

1. A tube cleaning apparatus for insertion through an interior of a fluid transfer tube having a discharge end removably insertable into a patient, comprising:
an elongated member including an insertion end and a manipulated end, said elongated member having a sufficient diameter and an axial length sufficiently sized for insertion through the interior of the fluid transfer tube having the discharge end inserted into the patient;
a resilient material disposed to extend radially from said insertion end, whereby said resilient material is extended radially a sufficient distance from said insertion end to provide contact against the interior of the fluid transfer tube upon insertion of said insertion end therethrough; and
a limit member removably clamped at any one of a plurality of positions along said axial length of said elongated member, said limit member is clamped at one of the plurality of positions to limit insertion of said insertion end of said elongated member through the interior of the fluid transfer tube.

2. The tube cleaning apparatus of claim 1 wherein said insertion end includes a distal portion having a pliable cylindrical tip disposed thereon.

3. The tube cleaning apparatus of claim 2 wherein said resilient material on said insertion end includes a plurality of filaments disposed to extend radially in increasing diameter from said distal portion of said insertion end.

4. The tube cleaning apparatus of claim 2 wherein said resilient material of said insertion end is composed of a porous flexible material having a conical shape extended radially in increasing diameter from said distal portion of said insertion end.

5. The tube cleaning apparatus of claim 1 wherein said limit member includes two aligned segments pivotably attached at a hinge connecting one abutting end of each aligned segment, said limit member is manipulated between an open position and a closed position clamped around said elongated member, whereby said closed position of said clamped limit member is adjustable to position said insertion end within the discharge end of the fluid transfer tube.

6. The tube cleaning apparatus of claim 5 wherein said limit member includes means for connecting each aligned segment together, said means for connecting is manipulated to maintain said limit member in said closed position around said elongated member at one of the plurality of positions along the axial length of said elongated member, said closed position around said elongated member is selected to provide a length increment for said elongated member equal to an insertion length for the discharge end of the fluid transfer tube inserted into the patient.

7. The tube cleaning apparatus of claim 6 wherein said limit member is clamped in said closed position on said elongated member at said length increment for said elongated member with said insertion end positioned within the discharge end of the fluid transfer tube, whereby the interior of the fluid transfer tube is cleaned by physical contact of said resilient material disposed on said insertion end against the interior of the fluid transfer tube.

8. The tube cleaning apparatus of claim 1 wherein the fluid transfer tube includes a sufficient length for insertion of the discharge end into the patient, and the fluid transfer tube includes a tube selected from the group consisting of a feeding tube insertable through the mouth, a feeding tube insertable through the nasal passageway, and a percutaneous endogastric tube.

9. The tube cleaning apparatus of claim 1 wherein said elongated member includes a hollow internal core disposed along an axial length of said tube member between about said distal end of said insertion end to about said manipulated end.

10. A tube cleaning apparatus for insertion through an interior of a fluid transfer tube having a discharge end inserted into a patient's stomach, comprising:
an elongated member including an insertion end and a manipulated end, said elongated member having a sufficient diameter and an axial length sufficiently sized for insertion through the fluid transfer tube having the discharge end inserted into the patient's stomach;
a resilient material disposed on said insertion end, said resilient material extending laterally from said insertion end for contact against the interior of the fluid transfer tube during insertion of said elongated member through the fluid transfer tube; and
a limit member is adjustably clamped at any of a plurality of positions along the axial length of said elongated member, said limit member provides an adjustable limit for insertion of said insertion end of said elongated member through the interior of the fluid transfer tube.

11. The tube cleaning apparatus of claim 10 wherein said elongated member includes a distal portion of said insertion end having a pliable cylindrical tip disposed proximal of said insertion end, and includes a hollow internal core disposed along an axial length of said elongated member between about said distal end of said insertion end to about said manipulated end.

12. The tube cleaning apparatus of claim 11 wherein said resilient material on said insertion end includes a conical shape extended radially in increasing diameter from said distal portion of said insertion end, said resilient material is selected from the group consisting of a porous flexible material and a plurality of filaments.

13. The tube cleaning apparatus of claim 10 wherein said limit member is clamped at one of the plurality of positions along the axial length of said elongated member to provide an appropriate length increment of said elongated member substantially equal to an insertion length of the fluid transfer tube having the discharge end inserted into the patient's stomach, whereby said clamped limit member limits insertion of said insertion end through said fluid transfer tube to no more than said appropriate length increment of said elongated member.

14. A tube cleaning apparatus for insertion through an interior of a percutaneous endogastric tube having a discharge end inserted into a patient's stomach, comprising:
an elongated member including an insertion end and a manipulated end, said elongated member having a sufficient diameter and an axial length sufficiently sized for insertion through the percutaneous endogastric tube having the discharge end inserted into the patient's stomach;
a resilient material disposed on said insertion end, said resilient material is laterally extended from a perimeter of said insertion end for contact against the percutaneous endogastric tube interior during insertion of said elongated member through the percutaneous endogastric tube when inserted into the patient; and
a limit member adjustably clamped at any one of a plurality of positions along the axial length of said elongated member, said clamped limit member provides an adjustable limit for insertion of said insertion end of said elongated member through the interior of the percutaneous endogastric tube.

15. The tube cleaning apparatus of claim 14 wherein said elongated member includes a distal portion of said insertion end having a pliable cylindrical tip disposed proximal of said insertion end, and includes a hollow internal core disposed along an axial length of said elongated member between about said distal end of said insertion end to about said manipulated end.

16. The tube cleaning apparatus of claim 15 wherein said resilient material on said insertion end includes a conical shape extended radially in increasing diameter from said distal portion of said insertion end, said resilient material is selected from the group consisting of a porous flexible material and a plurality of filaments.

17. The tube cleaning apparatus of claim 14 wherein said limit member is clamped at an appropriate length increment of said elongated member equal to an insertion length for the fluid transfer tube inserted into the patient, whereby said clamped limit member limits insertion of said elongated member into the fluid transfer tube.

18. A method of cleaning an interior of a fluid transfer tube having a discharge end retained within a patient, comprising the steps of:
selecting an elongated tube member including an insertion end having a resilient member thereon, said step of selecting including selecting an appropriate length of said tube member for extending said insertion end through the interior of the fluid transfer tube and into the discharge end of the fluid transfer tube;
positioning said insertion end having said resilient member thereon into the fluid transfer tube;
extending said insertion end having said resilient member thereon through the interior of the fluid transfer tube and into the discharge end of the fluid transfer tube; and
removing said insertion end having said resilient member thereon from the interior of the fluid transfer tube.

19. The method of cleaning of claim 18 including:
said step of selecting further including selecting a limit member for removably clamping on said tube member; and
said step of positioning further including positioning said limit member at about said appropriate length of said tube member thereby allowing said insertion end to be extended into the discharge end of the fluid transfer tube;
whereby said step of positioning said limit member limits said step of extending said insertion end of said tube member into the discharge end of the fluid transfer tube retained within the patient.

20. The method of cleaning of claim 19 further including:
injecting a selected volume of fluid through the interior of the fluid transfer tube and into the discharge end of the fluid transfer tube, said selected volume of fluid providing a minimal additional volume of fluid transferred into the patient, whereby said step of injecting providing additional cleaning of the interior of the fluid transfer tube retained within the patient.
